(19) United States
(12) Patent Application Publication
(10) Pub. No.: US 2006/0111716 A1
(43) Pub. Date: May 25, 2006

(54) Intramedullary nail

(76) Inventors: Andre Schlienger, Arlesheim (CH); Markus Buettler, Mumliswil (CH); Peter Senn, Waldenburg (CH)

Correspondence Address: JONES DAY, 222 EAST 41ST STREET, NEW YORK, NY, 10017-6702, US

(21) Appl. No.: 11/233,254

(22) Filed: Sep. 21, 2005

Related U.S. Application Data
(63) Continuation of application No. PCT/CH03/00184, filed on Mar. 21, 2003.

(51) Int. Cl. A61B 17/56

(52) U.S. Cl. 606/64

(57) ABSTRACT

The intramedullary nail has a distal end suitable for insertion in the medullary space, wherein the segment of the intramedullary nail facing the distal end has a diameter D, a proximal end, a central longitudinal axis; and several cross holes with a hole axis, each of which defines a virtual borehole cylinder with a cylinder axis corresponding to the hole axis of the defining cross hole, wherein the borehole cylinders of at least two cross holes penetrate each other. The cylinder axes of the two mutually penetrating borehole cylinders do not have any common intersection point P on the longitudinal axis. This results in improved mechanical strength compared to the state of the art, without any need for the size of the cross section of the intramedullary nail to be increased.

### RELATED APPLICATION DATA

The present application is a continuation of the U.S. National Stage designation of co-pending International Patent Application No. PCT/CH2003/000184, filed Mar. 21, 2003, the entire content of which is expressly incorporated herein by reference thereto.

### FIELD OF INVENTION

The invention relates to an intramedullary nail for use in treating bone fractures, e.g., the distal tibia. More particularly, the present invention relates to an intramedullary nail having at least two intersecting transverse boreholes, wherein the axes of the two boreholes intersect at a point that is offset from the central longitudinal axis of the nail.

### BACKGROUND OF THE INVENTION

The state of the art already includes intramedullary nails that have two intersecting transverse boreholes at the same height, so that the same intramedullary nail can be used both for a right and for a left tubular bone.

The disadvantages of this prior known arrangement is that the two (or even more) holes intersect and their axis of intersection lies on the longitudinal axis of the nail, with the result that a relatively large quantity of material is taken from the cross section of the intramedullary nail, which in turn leads to considerable mechanical weakening of the intramedullary nail at this point.

The invention is intended to remedy this situation. The invention is based on the problem of creating an intramedullary nail with two (or more) intersecting transverse boreholes that has improved mechanical strength compared to the state of the art described above, but which does not require the size of the cross section of the intramedullary nail to be increased.

### SUMMARY OF THE INVENTION

The invention solves the specified task by providing an intramedullary nail having a proximal end, a distal end, a central longitudinal axis, and a plurality of transverse boreholes, each having a hole axis and defining a virtual borehole cylinder having a cylinder axis corresponding to the hole axis of the defining transverse borehole. The borehole cylinders of at least two of the transverse boreholes mutually penetrate one another and the cylinder axes of the two mutually penetrating borehole cylinders intersect at a point that does not lie on the central longitudinal axis of the nail.

One variation of this invention is that the cylinder axes of the two mutually penetrating borehole cylinders meet at an intersection point P which is at a distance d>0 from the longitudinal axis. The distance d relative to the diameter D of the intramedullary nail is advantageously in the range 0.0001 D<d<0.6000 D, preferably in the range of 0.2 D<d<0.5 D.

One further variation is that the cylinder axes of the two mutually penetrating borehole cylinders are skewed and have a shortest distance x between them, where x>0. The length x in this system should be less than half the total of the two diameters of the mutually penetrating borehole cylinders to enable the two borehole cylinders to be penetrated. The segment S of the length x representing the shortest distance between the two cylinder axes can run parallel or at an angle to the longitudinal axis, and the shortest distance y between the segment S and the longitudinal axis is greater than zero. The distance y is advantageously less than D/2, but greater than 0.4 D.

In a further embodiment, the cylinder axes of the two mutually penetrating borehole cylinders lie in a plane orthogonal to the longitudinal axis.

In another embodiment, the cylinder axes of the two mutually penetrating borehole cylinders lie in a plane that intersects the longitudinal axis at an angle α, wherein 0°<α<60° and preferably 0°<α<30°. The advantage of this slanted arrangement compared to the orthogonal arrangement is to be found in the potentially longer bearing length of the screw to be inserted into the borehole cylinder, which leads to greater stability. A further advantage is improved fracture treatment.

In a further embodiment, the cylinder axes of the two mutually penetrating borehole cylinders intersect at an angle β, wherein 30°<β<90°, and preferably 50°<β<80°. Selection of the ideal angle will lead to optimisation of the wall thickness of the intramedullary nail.

In a preferred embodiment, the mutually penetrating borehole cylinders are provided with two separate entrance points into the intramedullary nail, but only one common exit from the intramedullary nail. The advantage of this system compared to one with four openings is that this system has less material loss, i.e. greater strength of the intramedullary nail.

The diameter D_{B} of the mutually penetrating borehole cylinders advantageously measures at most 0.6 times, but preferably at most 0.5 times of D.

In a further embodiment, the intramedullary nail is provided with a longitudinal hole coaxial to the central longitudinal axis. The bigger the diameter is of this hollow relative to the diameters of the borehole cylinders, the greater can be the angle between the mutually penetrating cross holes, without any impairment to the cross section area of the intramedullary nail, i.e. without any lessening of the strength of the intramedullary nail.

### BRIEF DESCRIPTION OF THE DRAWINGS

The invention and refinements of the invention are described in more detail in the following on the basis of partially schematic illustrations of several embodiments.

FIG. 1 shows a front and rear view of the intramedullary nail with two intersecting transverse boreholes;

FIG. 2 shows a front view of the intramedullary nail with a schematic, perspective view of the borehole cylinders;

FIG. 3 shows an orthogonal cross section through the intramedullary nail according to FIG. 2 in the range of the intersecting transverse boreholes; and

FIG. 4 shows a schematic perspective view of two mutually penetrating borehole cylinders with skewed cylinder axes.

### DETAILED DESCRIPTION OF THE PREFERRED EMBODIMENTS

The intramedullary nail illustrated in FIGS. 1 to 3 is provided with a distal end 2 suitable for insertion in the medullary space of a tubular bone, a proximal end 3, a central longitudinal axis 4 and two cross holes 5 with a hole axis 6, each of which defines a virtual borehole cylinder 8 with a cylinder axis 9 corresponding to the hole axis 6 of the defining cross hole 5. The intramedullary nail has generally a constant diameter D and a longitudinal hole 10 coaxial to the central longitudinal axis 4.

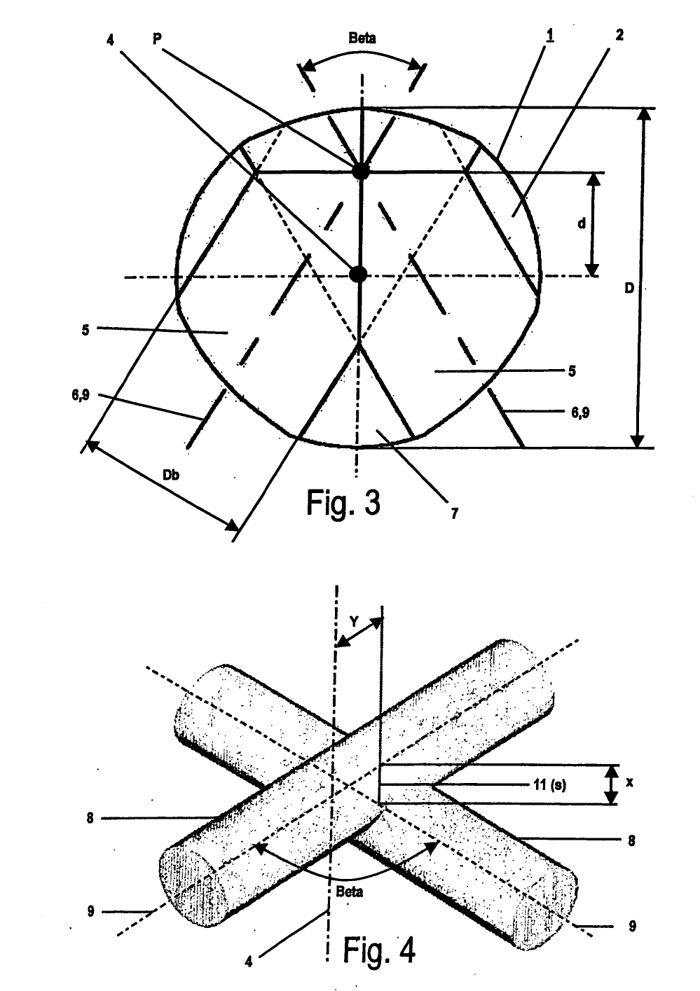

As illustrated in FIG. 3, the borehole cylinders 8 of the two cross holes 5 penetrate each other, wherein the cylinder axes 9 of the two mutually penetrating borehole cylinders 8 intersect at point P, which is at a distance d=0.4 D from the longitudinal axis 4. In other words, the point P does not lie on the longitudinal axis 4 of the intramedullary nail.

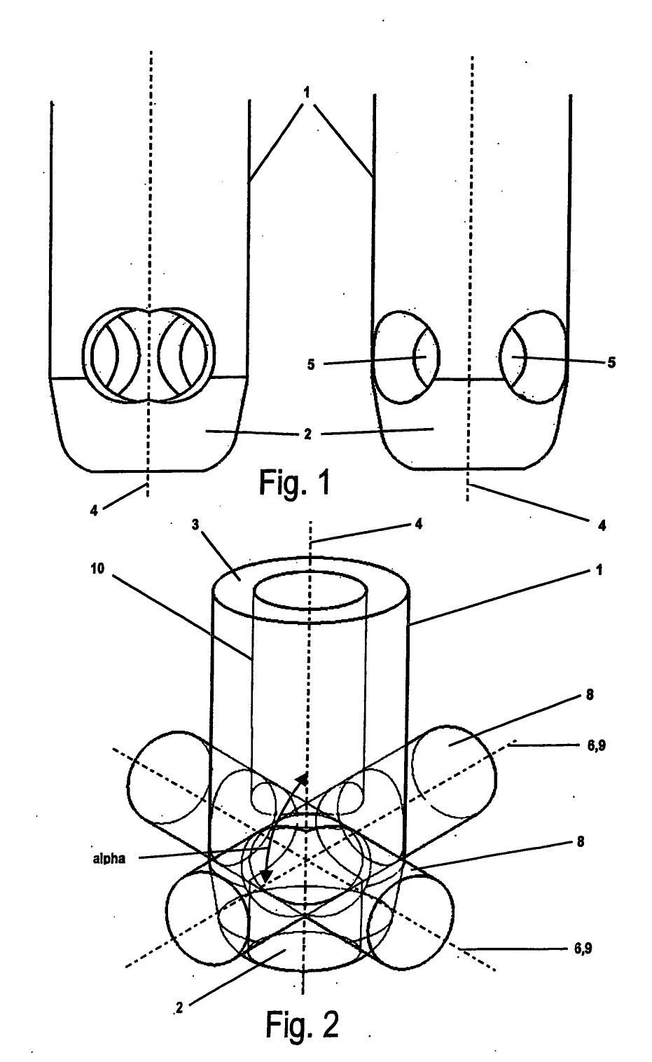

The mutually penetrating borehole cylinders 8 are provided with two separate entrance points in the intramedullary nail 1, but only one common exit from the intramedullary nail 1. The cylinder axes 9 of the two mutually penetrating borehole cylinders 8 lie in a plane orthogonal to the longitudinal axis 4, corresponding to the plane of projection of FIG. 3. The cylinder axes 9 of the two mutually penetrating borehole cylinders 8 can, however, also be in one plane, which is penetrated by the longitudinal axis 4 at a defined angle α (FIG. 2).

In the illustrated example, the cylinder axes 9 of the two mutually penetrating borehole cylinders 8 intersect at an angle β of 60°.

The diameter D_{B} of the mutually penetrating borehole cylinders 8 measures in the illustrated example 0.3 times D.

As illustrated in FIG. 4, the cylinder axes 9 of the two mutually penetrating borehole cylinders 8 in an alternative embodiment can also be skewed and the shortest distance 11 between them has a length x, which is smaller than half the total of the two diameters of the mutually penetrating borehole cylinders 8. The segment S of the length x representing the shortest distance 11 between the two cylinder axes 9 runs in the illustrated example parallel to the longitudinal axis 4, where the shortest distance y between the segment S and the longitudinal axis 4 is greater than zero. The section S can, however, also form an angle with respect to the longitudinal axis 4. Preferably, the distance y is in the range of D/2>y>0.4 D.

What is claimed is:

1. An intramedullary nail comprising:
a nail body configured and dimensioned for insertion into a medullary space of a bone, the nail body having a proximal end, a distal end and a central longitudinal axis, wherein the distal end of the nail body has a diameter D;
a plurality of transverse boreholes, each having a hole axis and defining a virtual borehole cylinder having a cylinder axis corresponding to the hole axis of the defining transverse borehole
wherein the borehole cylinders of at least two of the transverse boreholes mutually penetrate one another and the cylinder axes of the two mutually penetrating borehole cylinders intersect at a point that does not lie on the central longitudinal axis of the nail.

2. The intramedullary nail of claim 1, wherein the cylinder axes of the two mutually penetrating borehole cylinders intersect at a point P, which is at a distance d>0 from the central longitudinal axis of the nail.

3. The intramedullary nail of claim 1, wherein the cylinder axes of the two mutually penetrating borehole cylinders are offset, such that the shortest distance between them has a length x>0.

4. The intramedullary nail of claim 3, wherein each of the two mutually penetrating borehole cylinders has a diameter, and the length x is less than half the sum of the two diameters of the mutually penetrating borehole cylinders.

5. The intramedullary nail of claim 3, wherein a segment S of the length x representing the shortest distance between the two offset borehole cylinder axes runs parallel to the longitudinal axis of the nail, and the shortest distance y between segment S and the longitudinal axis is greater than zero.

6. The intramedullary nail of claim 5, wherein the distance y is less than D/2.

7. The intramedullary nail of claim 5, wherein the distance y is greater than 0.4D.

8. The intramedullary of claim 3, wherein a segment S of the length x representing the shortest distance between the two offset borehole cylinder axes is angled with respect to the longitudinal axis of the nail, and the shortest distance y between segment S and the longitudinal axis is greater than zero.

9. The intramedullary nail of claim 1, wherein the cylinder axes of the two mutually penetrating borehole cylinders lie in a plane orthogonal to the longitudinal axis of the nail.

10. The intramedullary nail of claim 1, wherein the cylinder axes of the two mutually penetrating borehole cylinders lie in a plane that intersects the longitudinal axis of the nail at an angle α, wherein 0°<α<60°.

11. The intramedullary nail of claim 1, wherein the cylinder axes of the two mutually penetrating borehole cylinders lie in a plane that intersects the longitudinal axis of the nail at an angle α, wherein 0°<α<30°.

12. The intramedullary nail of claim 1, wherein the cylinder axes of the two mutually penetrating borehole cylinders intersect at an angle β, wherein 30°<β<90°.

13. The intramedullary nail of claim 1, wherein the cylinder axes of the two mutually penetrating borehole cylinders intersect at an angle β, wherein 50°<β<80°.

14. The intramedullary nail of claim 2, wherein the distance d relative to the diameter D of the nail lies in the range 0.0001D<d<0.6000D.

15. The intramedullary nail of claim 2, wherein the distance d relative to the diameter D of the nail lies in the range 0.2D<d<0.5D.

16. The intramedullary nail of claim 1, wherein the two mutually penetrating borehole cylinders are provided with two separate entrance points into the intramedullary nail, but only one common exit from the intramedullary nail.

17. The intramedullary nail of claim 1, wherein the diameter D_{B} of the mutually penetrating borehole cylinders is not greater than 0.6 times the diameter D of the nail.

18. The intramedullary nail of claim 1, wherein the diameter D_{B} of the mutually penetrating borehole cylinders is not greater than 0.5 times the diameter D of the nail.

19. The intramedullary nail of claim 1, further comprising a longitudinal bore coaxial to the central longitudinal axis of the nail.

20. An intramedullary nail comprising:
a nail body configured and dimensioned for insertion into a medullary space of a bone, the nail body having a proximal end, a distal end and a central longitudinal axis, wherein the distal end of the nail body has a diameter D;
a plurality of transverse boreholes, each having a hole axis and defining a virtual borehole cylinder having a cylinder axis corresponding to the hole axis of the defining transverse borehole
wherein the borehole cylinders of at least two of the transverse boreholes mutually penetrate one another, and the cylinder axes of the two mutually penetrating borehole cylinders intersect and lie in a plane that intersects the longitudinal axis of the nail at an angle greater than zero.

21. The intramedullary nail of claim 20, wherein the cylinder axes of the two mutually penetrating borehole cylinders lie in a plane which is orthogonal to the longitudinal axis.

22. The intramedullary nail of claim 20, wherein the cylinder axes of the two mutually penetrating borehole cylinders lie in a plane that intersects the longitudinal axis of the nail at an angle α, wherein 0°<α<60°.

23. The intramedullary nail of claim 20, wherein the cylinder axes of the two mutually penetrating borehole cylinders intersect at an angle β, wherein 30°<β<90°.

24. The intramedullary nail of claim 20, wherein the two mutually penetrating borehole cylinders are provided with two separate entrance points into the intramedullary nail, but only one common exit from the intramedullary nail.
